(19) United States
(12) Patent Application Publication
(10) Pub. No.: US 2023/0401353 A1
(43) Pub. Date: Dec. 14, 2023

(54) METHOD AND SYSTEM FOR IDENTIFYING TIME DELAY IN EXTRACTION AND SEPARATION PROCESS OF RARE EARTH

(71) Applicant: EAST CHINA JIAOTONG UNIVERSITY, Nanchang (CN)

(72) Inventors: RONGXIU LU, NANCHANG (CN); HONGLIANG LIU, NANCHANG (CN); HUI YANG, NANCHANG (CN); JIANYONG ZHU, NANCHANG (CN); GANG YANG, NANCHANG (CN); WENJIE LI, NANCHANG (CN)

(21) Appl. No.: 17/899,346

(22) Filed: Aug. 30, 2022

(30) Foreign Application Priority Data
Jun. 13, 2022 (CN) 202210659488.3

(51) Int. Cl. G06F 30/20

(52) U.S. Cl. CPC G06F 30/20; G06F 2111/10

(57) ABSTRACT

A method and system for identifying a time delay in an extraction and separation process of rare earth: a reference sequence and comparison sequences are generated based on component contents of multiple rare earth elements and multiple process variables, and then preprocessed to determine a grey correlation. A comparison sequence with a highest correlation with the reference sequence is determined. An original data matrix is formed by taking the comparison sequence as a process variable. A time-correlation data matrix is constructed based on the obtained time delay sequence, time base sequence, and original data matrix to generate a time-correlation analysis matrix. A matrix H_{∞} norm is used to quantitatively describe the characteristics of the time-correlation analysis matrix, so as to determine a time delay sequence corresponding to a maximum H_{∞} norm as a to-be-solved multi-time delay.

### CROSS REFERENCE TO RELATED APPLICATION

This patent application claims the benefit and priority of Chinese Patent Application No. 202210659488.3, filed on Jun. 13, 2022, the disclosure of which is incorporated by reference herein in its entirety as part of the present application.

### TECHNICAL FIELD

The present disclosure relates to the technical field of rare earth extraction and separation, and in particular, to a method and system for identifying a time delay in an extraction and separation process of rare earth.

### BACKGROUND ART

Rare earth is an indispensable raw material for the development of high-tech industries such as advanced equipment manufacturing, new energy, and metamaterials, and strategic emerging industries. It also provides important support for the development and application of petrochemicals, electronic information, and metallurgy.

The extraction and separation process of rare earth is a typical non-linear industrial process with a large time delay. The extraction process is usually composed of dozens or even hundreds of extraction tanks connected in series. In addition, due to the different stirring rates and stirring times between each group of agitators, the reaction and transmission times of materials, extractants and detergents in the corresponding extraction tank groups are different, resulting in multi-time delay. Due to the multi-time delay, the system output cannot reflect the changes of the system input set value and control signal in time. Even if there is no time delay between the regulator and the regulating mechanism, it is necessary to go through the multi-time delay of the production process itself to cause the change of the regulated quantity, such that the regulation effect of the controller cannot act on the production process in real time. Due to the untimely regulation, the output overshoot of the system is large, and the regulation time is long, which leads to a longer transition process of the system and reduces the stability of the system. In addition, the existing modeling research of extraction process of rare earth does not consider the time delay or only substitutes the time delay as a constant, resulting in a certain gap between the established model and the actual rare earth extraction industry. The above phenomena directly or indirectly affect the quality of products and control, resulting in a lot of waste of energy and resources.

### SUMMARY

In order to solve the above problems existing in the prior art, the present disclosure provides a method and system for identifying a time delay in an extraction and separation process of rare earth.

In order to achieve the above objective, the present disclosure provides the following technical solutions:

A method for identifying a time delay in an extraction and separation process of rare earth includes:

obtaining a time delay sequence and a time base sequence;
generating a reference sequence based on component contents of multiple rare earth elements, and generating comparison sequences based on multiple process variables;
preprocessing the reference sequence and the comparison sequences to obtain preprocessed data;
obtaining a grey correlation according to the preprocessed data;
determining a comparison sequence with a highest correlation with the reference sequence based on the grey correlation;
forming an original data matrix by taking the comparison sequence with the highest correlation with the reference sequence as a process variable, where the original data matrix is A: A=[A₀, A₁, . . . A_{N}]; and in the formula, A₀ is a data sequence of an inlet process variable, Aᵢ is a data sequence of an outlet process variable of an i-th work unit, and i=1, 2, . . . , N;
constructing a time-correlation data matrix based on the time delay sequence, the time base sequence, and the original data matrix;
generating a time-correlation analysis matrix based on the time-correlation data matrix;
determining H_{∞} norms of the time-correlation analysis matrix; and
determining a time delay sequence corresponding to a maximum H_{∞} norm as a to-be-solved multi-time delay.

Preferably, a process of obtaining a grey correlation according to the preprocessed data may specifically include:
determining a correlation coefficient between an i-th process variable and a component content of a j-th rare earth element; and
determining a correlation between each process variable and a component content of each rare earth element according to the correlation coefficient, and taking the correlation as the grey correlation.

Preferably, a process of constructing a time-correlation data matrix based on the time delay sequence, the time base sequence, and the original data matrix may specifically include:
from a time t, selecting F continuous sampling data from the data sequence of the inlet process variable A₀ to obtain a first data time sequence, where the first data time sequence is x₀: x₀=[x_{0,t}, x_{0,t+T}, . . . , x_{0,t+fT}, . . . , x_{0,t+(F-1)T}]^{T};
determining a time delay between two adjacent data sequences in the original data matrix based on the time delay sequence;
based on the time delay between two adjacent data sequences, obtaining a second data time sequence in sequence, where the second data time sequence is xᵢ: xᵢ=[x_{i,t+τ}_{₁}_{+ . . . +τ}_{ᵢ}_{+T}, . . . , x_{i,t+τ}_{₁}_{+ . . . +τ}_{ᵢ}_{+jT}, . . . , x_{i,t+τ}_{₁}_{+ . . . +τ}_{ᵢ}_{+(F-1)T}]^{T}; and
constructing the time-correlation data matrix based on the first data time sequence and the second data time sequence, where the time-correlation data matrix is X:

$$X = [ \begin{matrix}{x}_{0 , t} & {x}_{1 , t + {τ}_{1}} & … & {x}_{N , t + {τ}_{1} + … + {τ}_{N}} \\ {x}_{0 , t + T} & {x}_{1 , t + {τ}_{1} + T} & … & {x}_{N , t + {τ}_{1} + … + {τ}_{N} + T} \\ ⋮ & ⋮ & ⋱ & ⋮ \\ {x}_{0 , t + ( F - 1 ) T} & {x}_{1 , t + {τ}_{1} + ( F - 1 ) T} & … & {x}_{N , t + {τ}_{1} + … + {τ}_{N} + ( F - 1 ) T}\end{matrix} ] ,$$

where

$$F \geq {\sum }_{i = 1}^{N} {d}_{i} ,$$

dᵢ is a time base corresponding to a time delay of the i-th work unit, τᵢ is the time delay, T is a sampling period, and x*,* is sampling data.

Preferably, a process of generating a time-correlation analysis matrix based on the time-correlation data matrix may specifically include:
obtaining a covariance matrix and a standard deviation of the time-correlation data matrix; and
generating the time-correlation analysis matrix based on the covariance matrix and the standard deviation, where the time-correlation analysis matrix is Rₓ:

$${R}_{x} = \frac{cov ( X )}{{Π}_{i = 1}^{N} {σ}_{i}} ,$$

cov(X) is the covariance matrix of the time-correlation data matrix, and σᵢ is a standard deviation of an i-th column in the time-correlation data matrix.

According to the specific embodiments provided by the present disclosure, the present disclosure discloses the following technical effects:

According to the method for identifying a time delay in an extraction and separation process of rare earth provided by the present disclosure, a reference sequence and comparison sequences are generated based on component contents of multiple rare earth elements and multiple process variables, and then preprocessed to determine a grey correlation. A comparison sequence with a highest correlation with the reference sequence is determined based on the grey correlation. An original data matrix is formed by taking the comparison sequence as a process variable. A time-correlation data matrix is constructed based on the obtained time delay sequence, time base sequence, and original data matrix to generate a time-correlation analysis matrix. Finally, a matrix H_{∞} norm is used to quantitatively describe the characteristics of the time-correlation analysis matrix, so as to determine a time delay sequence corresponding to a maximum H_{∞} norm as a to-be-solved multi-time delay. The control of the extraction and separation process of rare earth based on the multi-time delay can significantly improve the quality of the extracted rare earth, solve the problem of the gap between the established model and the actual rare earth extraction industry in the prior art, and fill the gap in time delay identification in the field of extraction and separation of rare earth.

Corresponding to the method for identifying a time delay in an extraction and separation process of rare earth provided above, the present disclosure further provides the following implementation system.

A system for identifying a time delay in an extraction and separation process of rare earth includes:
a sequence obtaining module configured to obtain a time delay sequence and a time base sequence;
a sequence generation module configured to generate a reference sequence based on component contents of multiple rare earth elements, and generate comparison sequences based on multiple process variables;
a data preprocessing module configured to preprocess the reference sequence and the comparison sequences to obtain preprocessed data;
a correlation determination module configured to obtain a grey correlation according to the preprocessed data;
a comparison sequence selection module configured to determine a comparison sequence with a highest correlation with the reference sequence based on the grey correlation;
a first matrix construction module configured to form an original data matrix by taking the comparison sequence with the highest correlation with the reference sequence as a process variable, where the original data matrix is A: A=[A₀, A₁, . . . A_{N}]; and in the formula, A₀ is a data sequence of an inlet process variable, Aᵢ is a data sequence of an outlet process variable of an i-th work unit, and i=1, 2, . . . , N;
a second matrix construction module configured to construct a time-correlation data matrix based on the time delay sequence, the time base sequence, and the original data matrix;
a third matrix construction module configured to generate a time-correlation analysis matrix based on the time-correlation data matrix;
a norm determination module configured to determine H_{∞} norms of the time-correlation analysis matrix; and
a multi-time delay determination module configured to determine a time delay sequence corresponding to a maximum H_{∞} norm as a to-be-solved multi-time delay.

Preferably, the correlation determination module may include:
a correlation coefficient determination unit configured to determine a correlation coefficient between an i-th process variable and a component content of a j-th rare earth element; and
a correlation determination unit configured to determine a correlation between each process variable and a component content of each rare earth element according to the correlation coefficient, and take the correlation as the grey correlation.

Preferably, the third matrix construction module may include:
an obtaining unit configured to obtain a covariance matrix and a standard deviation of the time-correlation data matrix; and
a second matrix construction unit configured to generate the time-correlation analysis matrix based on the covariance matrix and the standard deviation, where the time-correlation analysis matrix is Rₓ:

$${R}_{x} = \frac{cov ( X )}{{Π}_{i = 1}^{N} {σ}_{i}} ,$$

cov(X) is the covariance matrix of the time-correlation data matrix, and σᵢ is a standard deviation of an i-th column in the time-correlation data matrix.

Since the technical effect achieved by the system for identifying a time delay in an extraction and separation process of rare earth provided by the present disclosure is the same as the technical effect achieved by the method for identifying a time delay in an extraction and separation process of rare earth provided above, it will not be repeated here.

### BRIEF DESCRIPTION OF THE DRAWINGS

To describe the technical solutions in the embodiments of the present disclosure or in the prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is a flow chart of a method for identifying a time delay in an extraction and separation process of rare earth provided by the present disclosure;

FIG. 2 is a time delay identification result diagram of a 25-stage extraction tank provided by an embodiment of the present disclosure;

FIG. 3 is a comparison diagram of component content prediction errors of the 25-stage extraction tank provided by the embodiment of the present disclosure; and

FIG. 4 is a schematic diagram of a system for identifying a time delay in an extraction and separation process of rare earth provided by the present disclosure.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

An objective of the present disclosure is to provide a method and system for identifying a time delay in an extraction and separation process of rare earth, which can solve the problem of the gap between the established model and the actual rare earth extraction industry in the prior art, so as to significantly improve the quality of the extracted rare earth and fill the gap in time delay identification in the field of extraction and separation of rare earth.

To make the above-mentioned objective, features, and advantages of the present disclosure clearer and more comprehensible, the present disclosure will be further described in detail below in conjunction with the accompanying drawings and specific embodiments.

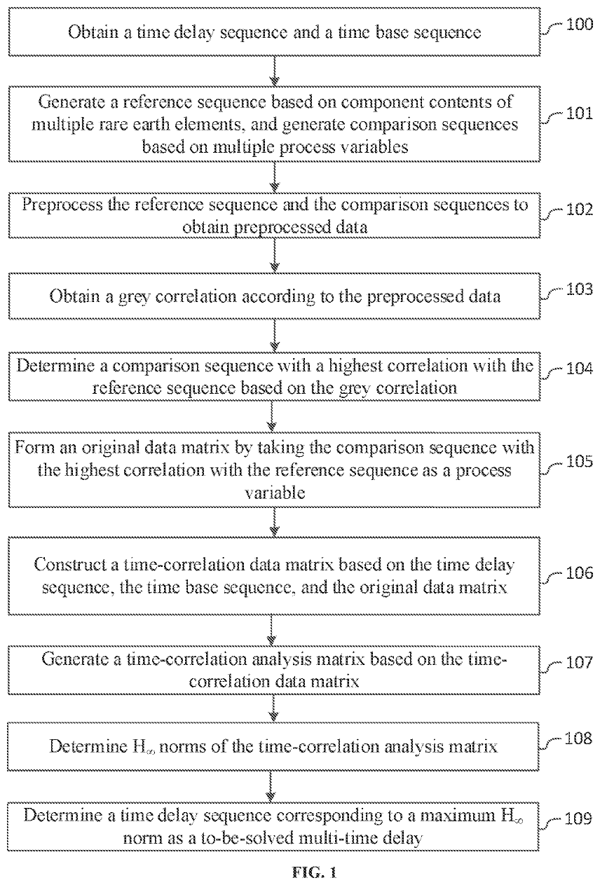

As shown in FIG. 1, a method for identifying a time delay in an extraction and separation process of rare earth provided by the present disclosure includes the following steps.

Step 100: A time delay sequence and a time base sequence are obtained. The construction process of the time delay sequence and the time base sequence is as follows.

A sampling period is set to be T, and a time delay sequence of a certain process variable of the extraction and separation process of the rare earth in N work units is recorded as: Γ=[τ₁, τ₂, . . . , τᵢ, . . . , τ_{N}]. In the formula, i=1, 2, . . . , N, τᵢ=dᵢT, and τᵢ is a time delay of an i-th work unit.

Then the time base sequence is: d=[d₁, d₂, . . . , dᵢ, . . . , d_{N}]. In the formula, is a time base corresponding to the time delay of the i-th unit, which is a dimensionless integer.

Step 101: A reference sequence is generated based on component contents of multiple rare earth elements, and comparison sequences are generated based on multiple process variables. For example, the component contents of n rare earth elements and data of m process variables are obtained by k sampling, and the component content of rare earth elements is taken as the reference sequence during correlation analysis: Uⱼ(t)=[u₀₁(t), u₀₂(t), . . . , u₀ⱼ(t), . . . , u₀ₙ(t)]. In the formula, 1≤j≤n, 1≤t≤k, an u₀ⱼ(t) represents a component content of a j-th rare earth element. The data of the process variable is taken as the comparison sequence: Uᵢ(t)=[u₁(t), . . . , uᵢ(t), . . . , uₘ(t)]. In the formula, 1≤i≤m, 1≤t≤k, and uᵢ(t) represents data of an i-th process variable.

Step 102: The reference sequence and the comparison sequences are preprocessed to obtain preprocessed data. The calculation of data preprocessing is as follows:

$${U}_{j}^{′} ( t ) = {U}_{j} ( t ) / \frac{1}{k} {\sum }_{t = 1}^{k} {U}_{j} ( t ) ,$$

$$and {U}_{i}^{′} ( t ) = {U}_{i} ( t ) / \frac{1}{k} {\sum }_{t = 1}^{m} {U}_{i} ( t ) .$$

In the formula, Uⱼ(t) is the processed reference sequence data, and Uᵢ(t) is the processed comparison sequence data.

Step 103: A grey correlation is obtained according to the preprocessed data. For example, the correlation coefficient of the preprocessed data is calculated specifically as follows:

$${ξ}_{ij} ( t ) = \frac{{min}_{i} {min}_{j} \vert {U}_{j}^{′} ( t ) - {U}_{i}^{′} ( t ) \vert + ρ {max}_{i} {max}_{j} \vert {U}_{j}^{′} ( t ) - {U}_{i}^{′} ( t ) \vert}{\vert {U}_{j}^{′} ( t ) {U}_{i}^{′} ( t ) \vert + ρ {max}_{i} {max}_{j} \vert {U}_{j}^{′} ( t ) {U}_{i}^{′} ( t ) \vert} .$$

In the formula, ξᵢⱼ(t) is a correlation coefficient of the i-th process variable corresponding to the component content of the j-th rare earth element, and p is called a resolution coefficient. A smaller ρ indicates a larger resolution. Generally, ρ is in a value range of [0,1], usually 0.5.

A correlation rᵢⱼ between each process variable and a component content of each rare earth element can be obtained according to the solved correlation coefficient, and the specific calculation is:

$${r}_{i j} = \frac{1}{k} {\sum }_{t = 1}^{k} {ξ}_{ij} ( t ) .$$

Step 104: A comparison sequence with a highest correlation with the reference sequence is determined based on the grey correlation. The correlation is sorted by size. If r₁₁<r₂₁, it means that a correlation between the comparison sequence u₂(t) and a component content of a first rare earth element is higher than that of the comparison sequence u₁(t), and then the comparison sequence with the highest correlation with the reference sequence can be selected.

Step 105: An original data matrix is formed by taking the comparison sequence with the highest correlation with the reference sequence as a process variable. The original data matrix is A: A=[A₀, A₁, . . . A_{N}]. In the formula, A₀ is a data sequence of an inlet process variable, Aᵢ is a data sequence of an outlet process variable of an i-th work unit, and i=1, 2, . . . , N.

Step 106: A time-correlation data matrix is constructed based on the time delay sequence, the time base sequence, and the original data matrix, which is specifically as follows.

From a time t, F continuous sampling data is selected from A₀ to obtain a data time sequence:

x₀=[x_{0,t}, x_{0,t+T}, . . . , x_{0,t+fT}, . . . , x_{0,t+(F-1)T}]^{T}.

In the formula,

$$F \geq {\sum }_{1}^{N} {d}_{i} ,$$

so as to ensure that the data in the time-correlation data matrix contains the information of the entire process cycle of the material flowing from the inlet to the outlet.

The time delay of A₁ relative to A₀ is τ₁, so the value principle is to take F continuous sampling data from A₁ from the time t+τ₁ to form the data time sequence x₁:

x₀=[x_{0,t+τ}_{₁}, x_{0,t+τ}_{₁}_{+T}, . . . , x_{0,t+τ}_{₁}_{+jT}, . . . , x_{0,t+τ}_{₁}_{+(F-1)T}]^{T}.

The rest of the work units are valued according to the above method and the corresponding time delay, namely:

xᵢ=[x_{i,t+τ}_{₁}_{+ . . . +τ}_{ᵢ}, x_{i,t+τ}_{₁}_{+ . . . +τ}_{ᵢ}_{+T}, . . . , x_{i,t+τ}_{₁}_{+ . . . +τ}_{ᵢ}_{+jT}, . . . , x_{i,t+τ}_{₁}_{+ . . . +τ}_{ᵢ}_{+(F-1)T}]^{T}.

Finally, the time-correlation data matrix constructed according to the time delay sequence is:

$$X = [ \begin{matrix}{x}_{0 , t} & {x}_{1 , t + {τ}_{1}} & … & {x}_{N , t + {τ}_{1} + … + {τ}_{N}} \\ {x}_{0 , t + T} & {x}_{1 , t + {τ}_{1} + T} & … & {x}_{N , t + {τ}_{1} + … + {τ}_{N} + T} \\ ⋮ & ⋮ & ⋱ & ⋮ \\ {x}_{0 , t + ( F - 1 ) T} & {x}_{1 , t + {τ}_{1} + ( F - 1 ) T} & … & {x}_{N , t + {τ}_{1} + … + {τ}_{N} + ( F - 1 ) T}\end{matrix} ] .$$

Step 107: A time-correlation analysis matrix is generated based on the time-correlation data matrix. The time-correlation between multiple time sequences is described by the time-correlation analysis matrix. The time-correlation analysis matrix is Rₓ:

$${R}_{x} = \frac{cov ( X )}{{Π}_{i = 1}^{N} {σ}_{i}} .$$

In the formula, cov(X) is the covariance matrix of the solved time-correlation data matrix X, and τᵢ is a standard deviation of an i-th column in the time-correlation data matrix X.

Step 108: H_{∞} norms of the time-correlation analysis matrix are determined.

Step 109: A time delay sequence corresponding to a maximum H_{∞} norm is determined as a to-be-solved multi-time delay.

Specifically, the matrix H_{∞} norm is used to quantitatively describe the characteristics of the time-correlation analysis matrix. The H_{∞} norm of the time-correlation analysis matrix Rₓ is solved, and its maximum H_{∞} norm is set to be β: β≤max(∥Rₓ∥_{∞}).

When the H_{∞} norm takes the maximum value β, the corresponding time delay sequence Γ is the to-be-solved multi-time delay.

In the present embodiment, the work unit is an extraction tank, the time base sequence is a dimensionless integer, and the time delay sequence is an integer multiple of the time base sequence.

The following takes the 25-stage praseodymium/neodymium extraction and separation production process of a rare earth extraction and separation enterprise as an example, and time delay identification is performed based on the method for identifying a time delay in an extraction and separation process of rare earth provided above.

In the industrial production process of praseodymium/neodymium cascade extraction, the content of praseodymium/neodymium components in different tanks will change with time, resulting in color changes. Therefore, a process variable with a color characteristic is selected to identify the time delay. The results of grey relational analysis (GRA) are shown in Table 1. The B component has the highest correlation, and the H component has the lowest correlation. Therefore, the B component data is used as the process variable, and 190 sets of data for continuous and stable production with a sampling period of 5 min are selected. Since every 5-stage extraction tank shares a set of agitators in the actual industrial site, it can be considered that every 5-stage extraction tank is a unit group, and the 25-stage extraction tank is constructed into 5 groups of units for identification. According to the flow direction of the extractant, the inlet sampling data and each group of outlet sampling data are recorded as a₀, a₁, a₂, a₃, a₄, and as respectively, so as to obtain the original data matrix A. Part of the original data matrix is shown in Table 2.

TABLE 1
GRA results

| Color characteristics | Correlation |
|---|---|
| R component | 0.6179 |
| G component | 0.5832 |
| B component | 0.6734 |
| H component | 0.543 |
| S component | 0.5706 |
| I component | 0.6123 |

TABLE 2
Part of original data matrix

| Original | | | | | | |
|---|---|---|---|---|---|---|
| data matrix | a0 | a1 | a2 | a3 | a4 | a5 |
| A1 | 0.435964 | 0.245997 | 0.117898 | 0.1587 | 0.406889 | 0.572275 |
| | 0.398143 | 0.229298 | 0.119521 | 0.16802 | 0.419837 | 0.554473 |
| | 0.368231 | 0.209023 | 0.121992 | 0.179546 | 0.436407 | 0.552915 |
| | 0.352368 | 0.185867 | 0.124394 | 0.189421 | 0.452121 | 0.566794 |
| | 0.347664 | 0.162072 | 0.125489 | 0.193597 | 0.46248 | 0.595304 |
| A2 | 0.435964 | 0.245997 | 0.117898 | 0.1587 | 0.406889 | 0.637635 |
| | 0.398143 | 0.229298 | 0.119521 | 0.16802 | 0.419837 | 0.691137 |
| | 0.368231 | 0.209023 | 0.121992 | 0.179546 | 0.436407 | 0.748547 |
| | 0.352368 | 0.185867 | 0.124394 | 0.189421 | 0.452121 | 0.80189 |
| | 0.347664 | 0.162072 | 0.125489 | 0.193597 | 0.46248 | 0.84319 |
| A3 | 0.435964 | 0.245997 | 0.117898 | 0.1587 | 0.406889 | 0.864498 |
| | 0.398143 | 0.229298 | 0.119521 | 0.16802 | 0.419837 | 0.86245 |
| | 0.368231 | 0.209023 | 0.121992 | 0.179546 | 0.436407 | 0.843418 |
| | 0.352368 | 0.185867 | 0.124394 | 0.189421 | 0.452121 | 0.815014 |
| | 0.347664 | 0.162072 | 0.125489 | 0.193597 | 0.46248 | 0.784853 |

According to field experience, the time delay between each stage of the extraction and separation process is in the range of [3,8] min. In view of the above construction, the time delay per unit group is in the range of [15,40] min. Therefore, the value range of the time base sequence is [3,8]. According to the above-constructed time-correlation data matrix X, the solution of the time delay sequence is quantized to the maximum H_{∞} norm.

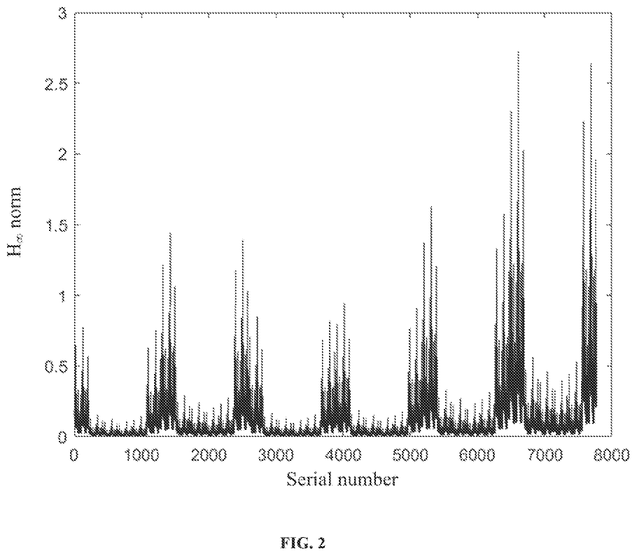

The enumeration method is used to find the maximum H_{∞} norm. FIG. 2 shows the result of the enumeration method. The maximum H_{∞} norm is 2.7287, and the corresponding time base sequence is [8 3 6 6 6]. Since the sampling period is 5 min, the time delay identified by the five unit groups is [40 15 30 30 30], that is, the time delay of the 25-stage praseodymium/neodymium extraction and separation production process is [8 8 8 8 8 3 3 3 3 3 6 6 6 6 6 6 6 6 6 6 6 6 6 6 6].

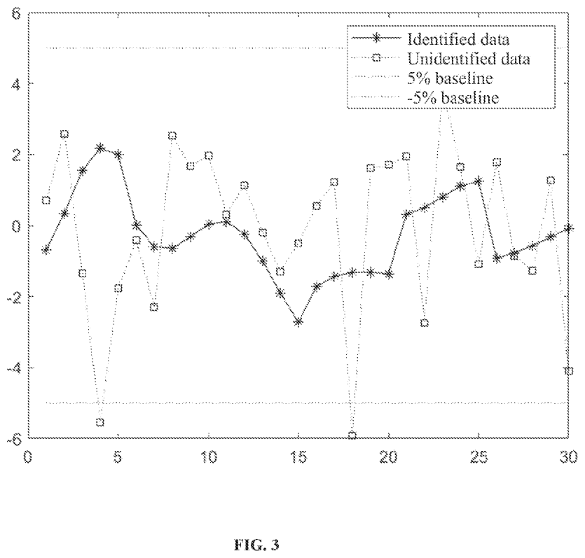

In order to verify the feasibility of the time delay identification method provided by the present disclosure, the identified data and the unidentified data are used for verification under the same prediction model through the wavelet neural network. It can be seen from Table 3 and FIG. 3 that the prediction indicators of the identified data are all optimal, and the maximum relative error is less than 5%, which meets the actual requirements, verifying the feasibility of the time delay identification method.

TABLE 3
Comparison of results before and after identification

| | Maximum relative | Average relative | Average absolute |
|---|---|---|---|
| Data | error % | error % | error % |
| Identified | 2.73 | 0.94 | 0.6237 |
| data | | | |
| Unidentified | 5.91 | 1.86 | 1.203 |
| data | | | |

Compared with the prior art, the present disclosure has the following significant advantages:

1. The method of the present disclosure fills the gap in time delay identification in the field of extraction and separation of rare earth.

2. The present disclosure can provide a new idea for the research on the modeling of the subsequent extraction and separation process of rare earth. Based on the present disclosure, the time delay of each stage of the extraction process of rare earth is identified, and is used to solve the problem that the time delay is not considered or the time delay is only substituted as a constant, resulting in a certain gap between the established model and the actual rare earth extraction industry in the mathematical model in the current extraction process of rare earth, so as to improve the modeling effect and reduce the modeling error.

3. The present disclosure can effectively utilize a large amount of data in the rare earth extraction and separation industrial site. Time delay identification can not only filter out the data that best matches the actual industrial process, but also match different types of data, that is, after using a certain type of data for identification, the mathematical model can be used to obtain the value of another type of data under the time delay.

4. The present disclosure can improve the effectiveness of industrial field control. In the extraction and separation process of rare earth, the control amount is usually operated with a certain size. The component content of rare earth elements measured at the outlet of the extraction tank actually reflects the change of the component content of rare earth elements before the lag time of the extraction tank in this section. Through the time delay identification, the real component content of rare earth elements at the outlet of the extraction tank can be deduced, so as to adjust the control amount in a targeted manner, thereby reducing the waste of raw materials in production, enabling rare earth extraction and separation enterprises to save energy, reduce consumption, increase production and increase efficiency, and improving the competitiveness and sustainable development of enterprises.

Corresponding to the method for identifying a time delay in an extraction and separation process of rare earth provided above, the present disclosure further provides the following implementation system.

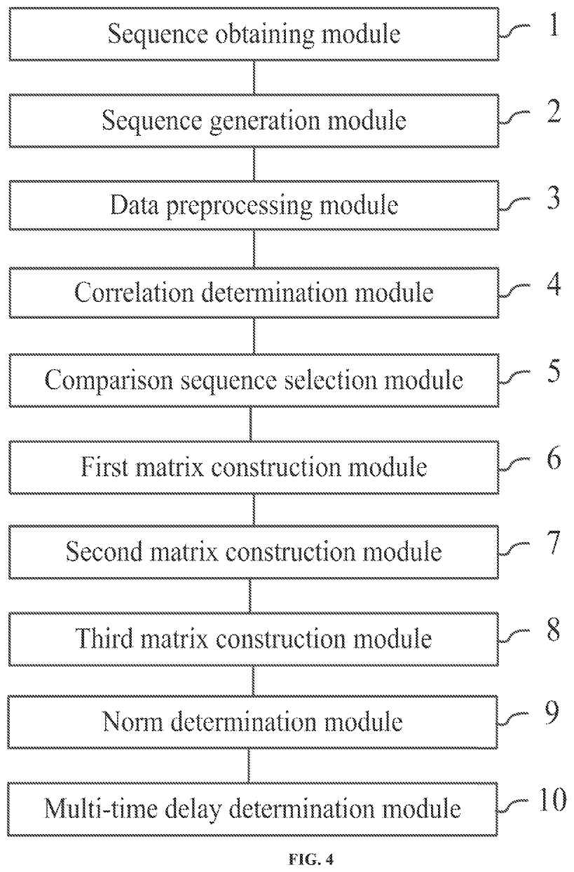

A system for identifying a time delay in an extraction and separation process of rare earth, as shown in FIG. 4, includes: a sequence obtaining module 1, a sequence generation module 2, a data preprocessing module 3, a correlation determination module 4, a comparison sequence selection module 5, a first matrix construction module 6, a second matrix construction module 7, a third matrix construction module 8, a norm determination module 9, and a multi-time delay determination module 10.

The sequence obtaining module 1 is configured to obtain a time delay sequence and a time base sequence.

The sequence generation module 2 is configured to generate a reference sequence based on component contents of multiple rare earth elements, and generate comparison sequences based on multiple process variables.

The data preprocessing module 3 is configured to preprocess the reference sequence and the comparison sequences to obtain preprocessed data.

The correlation determination module 4 is configured to obtain a grey correlation according to the preprocessed data.

The comparison sequence selection module 5 is configured to determine a comparison sequence with a highest correlation with the reference sequence based on the grey correlation.

The first matrix construction module 6 is configured to form an original data matrix by taking the comparison sequence with the highest correlation with the reference sequence as a process variable. The original data matrix is A: A=[A₀, A₁, . . . A_{N}]. In the formula, A₀ is a data sequence of an inlet process variable, Aᵢ is a data sequence of an outlet process variable of an i-th work unit, and i=1, 2, . . . , N.

The second matrix construction module 7 is configured to construct a time-correlation data matrix based on the time delay sequence, the time base sequence, and the original data matrix.

The third matrix construction module 8 is configured to generate a time-correlation analysis matrix based on the time-correlation data matrix.

The norm determination module 9 is configured to determine H_{∞} norms of the time-correlation analysis matrix.

The multi-time delay determination module 10 is configured to determine a time delay sequence corresponding to a maximum H_{∞} norm as a to-be-solved multi-time delay.

The correlation determination module 4 includes: a correlation coefficient determination unit and a correlation determination unit.

The correlation coefficient determination unit is configured to determine a correlation coefficient between an i-th process variable and a component content of a j-th rare earth element.

The correlation determination unit is configured to determine a correlation between each process variable and a component content of each rare earth element according to the correlation coefficient, and take the correlation as the grey correlation.

The third matrix construction module 8 includes: an obtaining unit and a second matrix construction unit.

The obtaining unit is configured to obtain a covariance matrix and a standard deviation of the time-correlation data matrix.

The second matrix construction unit is configured to generate the time-correlation analysis matrix based on the covariance matrix and the standard deviation. The time-correlation analysis matrix is Rₓ:

$${R}_{x} = \frac{cov ( X )}{{Π}_{i = 1}^{N} {σ}_{i}} ,$$

cov(X) is the covariance matrix of the time-correlation data matrix, and σᵢ is a standard deviation of an i-th column in the time-correlation data matrix.

Each embodiment of the present specification is described in a progressive manner, each embodiment focuses on the difference from other embodiments, and the same and similar parts between the embodiments may refer to each other. Since the system disclosed in an embodiment corresponds to the method disclosed in another embodiment, the description is relatively simple, and reference can be made to the method description.

Specific examples are used herein to explain the principles and embodiments of the present disclosure. The foregoing description of the embodiments is merely intended to help understand the method of the present disclosure and its core ideas; besides, various modifications may be made by those of ordinary skill in the art to specific embodiments and the scope of application in accordance with the ideas of the present disclosure. In conclusion, the content of the present description shall not be construed as limitations to the present disclosure.

What is claimed is:

1. A method for identifying a time delay in an extraction and separation process of rare earth, comprising:
obtaining a time delay sequence and a time base sequence;
generating a reference sequence based on component contents of multiple rare earth elements, and generating comparison sequences based on multiple process variables;
preprocessing the reference sequence and the comparison sequences to obtain preprocessed data;
obtaining a grey correlation according to the preprocessed data;
determining a comparison sequence with a highest correlation with the reference sequence based on the grey correlation;
forming an original data matrix by taking the comparison sequence with the highest correlation with the reference sequence as a process variable, wherein the original data matrix is A: A=[A₀, A₁, . . . A_{N}]; and in the formula, A₀ is a data sequence of an inlet process variable, A₁ is a data sequence of an outlet process variable of an i-th work unit, and i=1, 2, . . . , N;
constructing a time-correlation data matrix based on the time delay sequence, the time base sequence; and the original data matrix;
generating a time-correlation analysis matrix based on the time-correlation data matrix;
determining H_{∞} norms of the time-correlation analysis matrix; and
determining a time delay sequence corresponding to a maximum H_{∞} norm as a to-be-solved multi-time delay.

2. The method for identifying a time delay in an extraction and separation process of rare earth according to claim 1, wherein a process of obtaining a grey correlation according to the preprocessed data specifically comprises:
determining a correlation coefficient between an i-th process variable and a component content of a j-th rare earth element; and
determining a correlation between each process variable and a component content of each rare earth element according to the correlation coefficient, and taking the correlation as the grey correlation.

3. The method for identifying a time delay in an extraction and separation process of rare earth according to claim 2, wherein a process of constructing a time-correlation data matrix based on the time delay sequence, the time base sequence, and the original data matrix specifically comprises:
from a time t, selecting F continuous sampling data from the data sequence of the inlet process variable A₀ to obtain a first data time sequence, wherein the first data time sequence is x₀: x₀=[x_{0,t}, x_{0,t+T}, . . . , x_{0,t+fT}, . . . , x_{0,t+(F-1)T]}^{T};
determining a time delay between two adjacent data sequences in the original data matrix based on the time delay sequence;
based on the time delay between two adjacent data sequences, obtaining a second data time sequence in sequence, wherein the second data time sequence is xᵢ: xᵢ=[x_{i,t+τ}_{₁}_{+ . . . +τ}_{ᵢ}, x_{i,t+τ}_{₁}_{+ . . . +τ}_{ᵢ}_{+T}, . . . , x_{i,t+τ}_{₁}_{+ . . . +τ}_{ᵢ}_{+jT}, . . . , x_{i,t+τ}_{₁}_{+ . . . +τ}_{ᵢ}_{+(F-1)T}]^{T}; and
constructing the time-correlation data matrix based on the first data time sequence and the second data time sequence, wherein the time-correlation data matrix is X:
$$X = [ \begin{matrix}{x}_{0 , t} & {x}_{1 , t + {τ}_{1}} & … & {x}_{N , t + {τ}_{1} + … + {τ}_{N}} \\ {x}_{0 , t + T} & {x}_{1 , t + {τ}_{1} + T} & … & {x}_{N , t + {τ}_{1} + … + {τ}_{N} + T} \\ ⋮ & ⋮ & ⋱ & ⋮ \\ {x}_{0 , t + ( F - 1 ) T} & {x}_{1 , t + {τ}_{1} + ( F - 1 ) T} & … & {x}_{N , t + {τ}_{1} + … + {τ}_{N} + ( F - 1 ) T}\end{matrix} ] ;$$
wherein
$$F \geq {\sum }_{i = 1}^{N} {d}_{i} ,$$
dᵢ is a time base corresponding to a time delay of the i-th work unit, τᵢ is the time delay, T is a sampling period, and x*,* is sampling data.

4. The method for identifying a time delay in an extraction and separation process of rare earth according to claim 3, wherein a process of generating a time-correlation analysis matrix based on the time-correlation data matrix specifically comprises:
obtaining a covariance matrix and a standard deviation of the time-correlation data matrix; and
generating the time-correlation analysis matrix based on the covariance matrix and the standard deviation, wherein the time-correlation analysis matrix is Rₓ:
$${R}_{x} = \frac{cov ( X )}{{Π}_{i = 1}^{N} {σ}_{i}} ,$$
cov(X) is the covariance matrix of the time-correlation data matrix, and σᵢ is a standard deviation of an i-th column in the time-correlation data matrix.

5. A system for identifying a time delay in an extraction and separation process of rare earth, comprising:
a sequence obtaining module configured to obtain a time delay sequence and a time base sequence;
a sequence generation module configured to generate a reference sequence based on component contents of multiple rare earth elements, and generate comparison sequences based on multiple process variables;
a data preprocessing module configured to preprocess the reference sequence and the comparison sequences to obtain preprocessed data;
a correlation determination module configured to obtain a grey correlation according to the preprocessed data;
a comparison sequence selection module configured to determine a comparison sequence with a highest correlation with the reference sequence based on the grey correlation;
a first matrix construction module configured to form an original data matrix by taking the comparison sequence with the highest correlation with the reference sequence as a process variable, wherein the original data matrix is A: A=[A₀, A₁, . . . A_{N}]; and in the formula, A₀ is a data sequence of an inlet process variable, Aᵢ is a data sequence of an outlet process variable of an i-th work unit, and i=1, 2, . . . , N;
a second matrix construction module configured to construct a time-correlation data matrix based on the time delay sequence, the time base sequence, and the original data matrix;
a third matrix construction module configured to generate a time-correlation analysis matrix based on the time-correlation data matrix;
a norm determination module configured to determine H_{∞} norms of the time-correlation analysis matrix; and
a multi-time delay determination module configured to determine a time delay sequence corresponding to a maximum H_{∞} norm as a to-be-solved multi-time delay.

6. The system for identifying a time delay in an extraction and separation process of rare earth according to claim 5, wherein the correlation determination module comprises:
a correlation coefficient determination unit configured to determine a correlation coefficient between an i-th process variable and a component content of j-th rare earth element; and
a correlation determination unit configured to determine a correlation between each process variable and a component content of each rare earth element according to the correlation coefficient, and take the correlation as the grey correlation.

7. The system for identifying a time delay in an extraction and separation process of rare earth according to claim 6, wherein the third matrix construction module comprises:
an obtaining unit configured to obtain a covariance matrix and a standard deviation of the time-correlation data matrix; and
a second matrix construction unit configured to generate the time-correlation analysis matrix based on the covariance matrix and the standard deviation, wherein the time-correlation analysis matrix is Rₓ:
$${R}_{x} = \frac{cov ( X )}{{Π}_{i = 1}^{N} {σ}_{i}} ,$$
cov(X) is the covariance matrix of the time-correlation data matrix, and σᵢ is a standard deviation of an i-th column in the time-correlation data matrix.
